(19) United States
(12) Patent Application Publication
(10) Pub. No.: US 2022/0198596 A1
(43) Pub. Date: Jun. 23, 2022

(54) CARE SUPPORT APPARATUS, CARE SUPPORT METHOD AND CARE SUPPORT SYSTEM

(71) Applicant: ABA INC., Funabashi-shi (JP)

(72) Inventor: Yoshimi Ui, Funabashi-shi (JP)

(21) Appl. No.: 17/695,461

(22) Filed: Mar. 15, 2022

Related U.S. Application Data
(63) Continuation of application No. PCT/JP2020/035268, filed on Sep. 17, 2020.

(30) Foreign Application Priority Data
Sep. 24, 2019 (JP) 2019-173211

(51) Int. Cl. G06Q 50/22

(52) U.S. Cl. CPC G06Q 50/22; A61F 13/42

(57) ABSTRACT

A caregiving assistance device comprises: an excretion identification unit that identifies, on the basis of information from a sensor 1 provided around a person to be given care, an excretion time, which is the time at which the person to be given care has produced excrement; and an information output part that generates, on the basis of past excretion times of the person to be given care, recommendation information indicating when it is recommended that a caregiver carry out at least one caregiving task including disposal of the excrement of the person to be given care, and outputs the generated recommendation information.

### CROSS-REFERENCE TO RELATED APPLICATIONS

The present application is a continuation application of International Application number PCT/JP2020/35268, filed on Sep. 17, 2020, which claims priority under 35 U.S.C § 119(a) to Japanese Patent Application No. 2019-173211, filed on Sep. 24, 2019. The contents of these applications are incorporated herein by reference in their entirety.

### BACKGROUND OF THE INVENTION

The present disclosure relates to a care support apparatus, a care support method, and a care support system for supporting a caregiver who cares for a care recipient.

Japanese Unexamined Patent Application Publication No. 2018-143362 discloses an excretion detection system that detects that a care recipient, such as an elderly person who needs support in excretion treatment, has excreted.

When a care recipient excretes, it is desirable for the care recipient that this excretion be treated as soon as possible. However, since caregivers often have numerous duties, they are not always able to treat the excretion promptly.

### BRIEF SUMMARY OF THE INVENTION

The present disclosure focuses on this point, and an object of the present disclosure is to provide a care support apparatus, a care support method, a program, and a care support system that support a caregiver to treat excretion of a care recipient at an appropriate timing.

A first aspect of the present disclosure provides a care support apparatus that includes an excretion identification part that identifies an excretion time point at which a care recipient excretes on the basis of information from a sensor provided in the vicinity of the care recipient, and an information output part that generates recommendation information indicating when a caregiver of the care recipient is recommended to perform one or more types of care work including treatment of the excretion of the care recipient on the basis of past excretion time points of the care recipient, and outputs the generated recommendation information.

A second aspect of the present disclosure provides a care support method that includes the steps, executed by a computer, of identifying an excretion time point at which a care recipient excretes on the basis of information from a sensor provided in the vicinity of the care recipient, generating recommendation information indicating a time at which a caregiver of the care recipient is recommended to perform one or more types of care work including treatment of excretion of the care recipient on the basis of the past excretion time point of the care recipient, and outputting the generated recommendation information.

A third aspect of the present disclosure provides a care support system that includes an information terminal that is capable of displaying information, and a care support apparatus that outputs recommendation information to the information terminal, wherein the care support apparatus includes an excretion identification part that identifies an excretion time point at which a care recipient excretes on the basis of information from a sensor provided in the vicinity of the care recipient, and an information output part that generates recommendation information indicating a time at which a caregiver of the care recipient is recommended to perform one or more types of care work including treatment of excretion of the care recipient on the basis of past excretion time point of the care recipient, and outputs the generated recommendation information, and the information terminal includes a display part that displays the recommendation information.

### BRIEF DESCRIPTION OF THE DRAWINGS

FIG. 1 is a diagram for illustrating an overview of a care support system.

FIG. 2 shows a configuration of a sensor.

FIG. 3 shows a configuration of an information terminal.

FIG. 4 shows an example of a screen for inputting details of treatment of excretion of a care recipient, which is one type of care work.

FIG. 5 shows a configuration of a care support apparatus.

FIG. 6 shows an example of a care recipient database.

FIG. 7 shows an example of a screen including recommendation information outputted by an information output part.

FIG. 8 shows another example of a screen including recommendation information outputted by an information output part.

FIG. 9 is an example of a screen that makes it easier for a caregiver to keep track of excretion statuses of a plurality of care recipients.

FIG. 10 shows an example of a daily activity plan for a care recipient and caregiver outputted by the information output part.

FIG. 11 shows a history of the excretion times of a plurality of care recipients.

FIG. 12 shows the time of day when each care recipient is likely to excrete.

FIG. 13 shows available treatment times for each care recipient.

FIGS. 14A and 14B show available treatment times for each care recipient assigned to a plurality of caregivers.

FIG. 15 shows a configuration of a care support system according to a first variation.

FIG. 16 shows a configuration of a care support apparatus.

### DETAILED DESCRIPTION OF THE INVENTION

Hereinafter, the present invention will be described through exemplary embodiments of the present invention, but the following exemplary embodiments do not limit the invention according to the claims, and not all of the combinations of features described in the exemplary embodiments are necessarily essential to the solution means of the invention.

### [Overview of Care Support System S1]

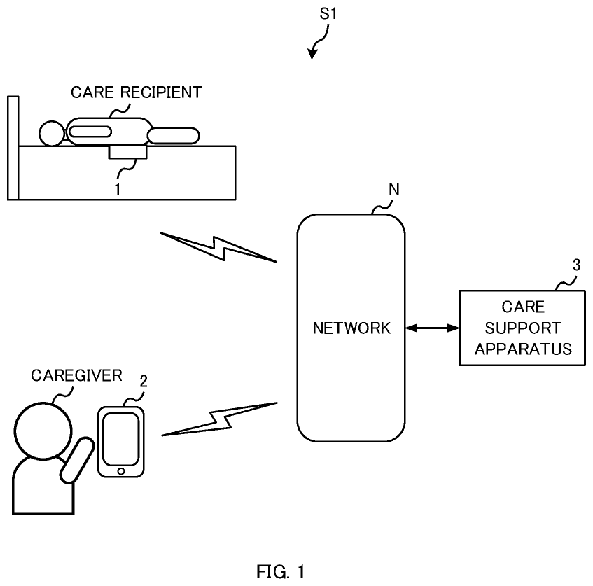

FIG. 1 is a diagram for illustrating an overview of a care support system S1. The care support system S1 is a system to support a caregiver's care work in a facility where a care recipient needs to be cared for, such as a care facility or hospital (hereinafter referred to as a “care facility or the like”). The care recipient is a person who is unable to treat excretion by himself/herself and wears a diaper, such as an elderly person who needs care. The caregiver is a person who performs care work such as treating the care recipient's excretion (that is, changing diapers), providing a meal for the care recipient, managing medication given to the care recipient, and helping the care recipient bathe.

It should be noted that, in the following embodiments, the description will be made using examples of the care support system S1 mainly intended to provide care support to care recipients such as elderly people, but the system is also applicable to the treatment of excretion for sick people and infants (that is, “care recipients” in the present disclosure can include not only so-called elderly care recipients but also sick people or infants).

The care support system S1 includes a sensor 1, an information terminal 2, and a care support apparatus 3. The sensor 1, the information terminal 2, and the care support apparatus 3 are connected to a network N. The network N is a wireless communication network installed in the care facility or the like. The sensor 1 and the information terminal 2 can transmit and receive various types of information to and from the care support apparatus 3 via the network N. Although only one sensor 1 and one information terminal 2 are shown in FIG. 1, the care support system S1 may include a plurality of sensors 1 and a plurality of information terminals 2.

The sensor 1 is a sensor that generates an odor signal that contains information according to the type and intensity of a detected odor. The sensor 1 is provided in the vicinity of the care recipient, such as the care recipient's bed or the care recipient's clothes. The sensor 1 generates odor detection information indicating the presence of a predetermined odor component in the vicinity of the care recipient, and transmits the odor detection information to the care support apparatus 3. The sensor 1 may, for example, transmit the odor detection information when the sensor 1 detects the odor component of a predetermined level or more, or the sensor 1 may transmit the odor detection information on a condition that the odor has changed by a certain level or more.

The information terminal 2 is an information terminal used by the caregiver, and is a tablet, a smartphone, or a personal computer, for example. The information terminal 2 receives an input of details of the care work performed by the caregiver, and displays information received from the care support apparatus 3.

The care support apparatus 3 is a computer, for example, and generates various types of information useful for supporting the caregiver's care work, on the basis of the odor signal transmitted from the sensor 1, and outputs the generated information. The information generated by the care support apparatus 3 is, for example, recommendation information for recommending to the caregiver contents of the care work to be performed or a time at which the care work should be performed. The recommendation information is not limited to information that describes the recommendation to the caregiver, but includes any information that is useful for the caregiver to perform the care work.

The care support apparatus 3 may generate information other than the recommendation to the caregiver. The care support apparatus 3 identifies a tendency of the excretion timing of the care recipient on the basis of, for example, the odor signal received from the plurality of sensors 1 provided in the bed, and generates information related to work sharing, staff planning, and the like in the care facility or the like on the basis of the identified tendency.

The care support apparatus 3 outputs the generated information in various ways. The care support apparatus 3 outputs information by, for example, displaying the generated information on a display, transmitting the information to an external apparatus such as an information terminal used by the caregiver, or causing a printer to print the information.

Configurations and operations of the sensor 1, the information terminal 2, and the care support apparatus 3 will be described in detail below.

### [Configuration of Sensor 1]

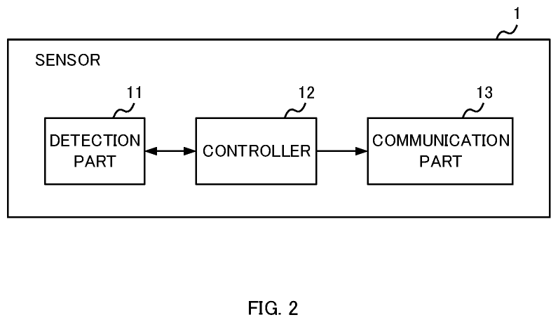

FIG. 2 shows a configuration of the sensor 1. The sensor 1 includes a detection part 11, a controller 12, and a communication part 13.

The detection part 11 generates the odor signal indicating the intensity of a specified odor component. The detection part 11 may generate the odor signal further indicating the type of odor. The detection part 11 inputs the generated odor signal to the controller 12.

The controller 12 generates the odor detection information on the basis of the odor signal inputted from the detection part 11. For example, if the level of the odor signal is equal to or higher than the predetermined level, the controller 12 generates the odor detection information that includes text data indicating that the odor is being detected. The controller 12 may generate the odor detection information that includes text data indicating that the odor has been detected if the level of the odor signal is greater than a predetermined value from the level indicating that no odor component has been detected.

The controller 12 may generate the odor detection information that includes data indicating the time together with text data indicating that the odor has been detected. Further, when the detection part 11 outputs the odor signal indicating the type of odor detected, the controller 12 may generate the odor detection information that includes text data indicating the type of odor. The controller 12 inputs the generated odor detection information to the communication part 13.

The communication part 13 includes a communication interface for transmitting the odor detection information inputted from the controller 12 to the care support apparatus 3 via the network N. The communication part 13 has a controller for transmitting the odor signal in a procedure defined by the Bluetooth (registered trademark) standard, for example, and transmits the odor signal to the care support apparatus 3 via a wireless router connected to the network N.

### [Configuration of Information Terminal 2]

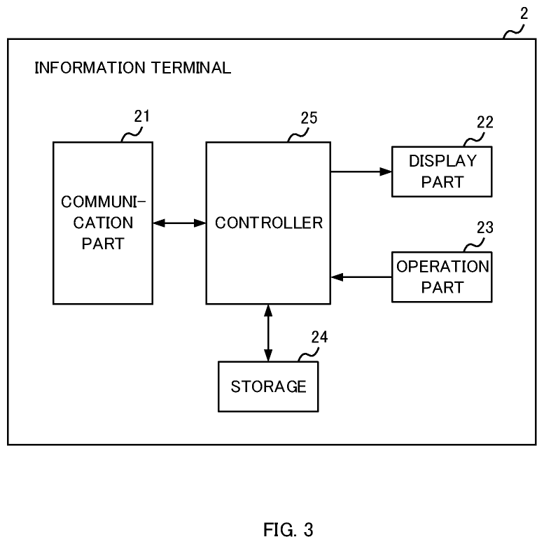

FIG. 3 shows a configuration of an information terminal 2. The information terminal 2 includes a communication part 21, a display part 22, an operation part 23, a storage 24, and a controller 25.

The communication part 21 includes a communication interface for transmitting and receiving various types of information to and from the care support apparatus 3 via the network N. The communication part 21 has a controller for transmitting and receiving information in a procedure defined by the Wi-Fi (registered trademark) standard, for example, and transmits and receives the information to and from the care support apparatus 3 via a wireless router connected to the network N.

The display part 22 displays a screen for inputting the contents of the care work performed by the caregiver, and displays various types of information such as the recommendation information received from the care support apparatus 3. The display part 22 may display various screens by executing application software installed in the information terminal 2 by the controller 25, or may display a screen transmitted from the care support apparatus 3.

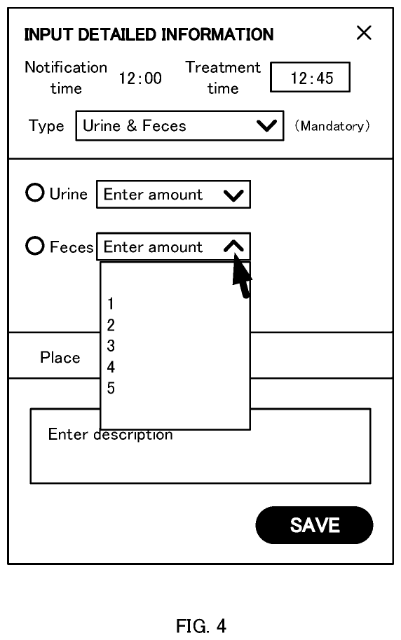

FIG. 4 shows an example of a screen for inputting the contents of the treatment of excretion of the care recipient, which is one type of the care work. In the example shown in FIG. 4, the screen is displayed for the caregiver to input whether the excretion is feces or urine, the amount of excretion, and a treatment time that is a time when the diaper was changed.

The operation part 23 is a device for receiving an operation of the caregiver who is a user of the information terminal 2, and is a touch panel provided on the display part 22, for example. The operation part 23 notifies the controller 25 of the received operation contents.

The storage 24 is a storage medium including a Read Only Memory (ROM) and a Random Access Memory (RAM). The storage 24 stores a program executed by the controller 25. The storage 24 stores various types of information such as the recommendation information received from the care support apparatus 3.

The controller 25 includes a processor such as a Central Processing Unit (CPU). By executing the program stored in the storage 24, the controller 25 displays the recommendation information received from the care support apparatus 3 on the display part 22, identifies the contents of the operation performed by the caregiver on the operation part 23, and executes a process corresponding to the identified contents. When the caregiver inputs that the diaper has been changed, for example, the controller 25 generates care treatment performance information indicating that treatment such as changing the diaper has been performed, and transmits the information to the care support apparatus 3 via the communication part 21. The care treatment performance information may include a state of excretion inputted by the caregiver. The state of excretion is, for example, the type of excretion (feces or urine), the amount of excretion, or the color of excretion.

### [Configuration of Care Support Apparatus 3]

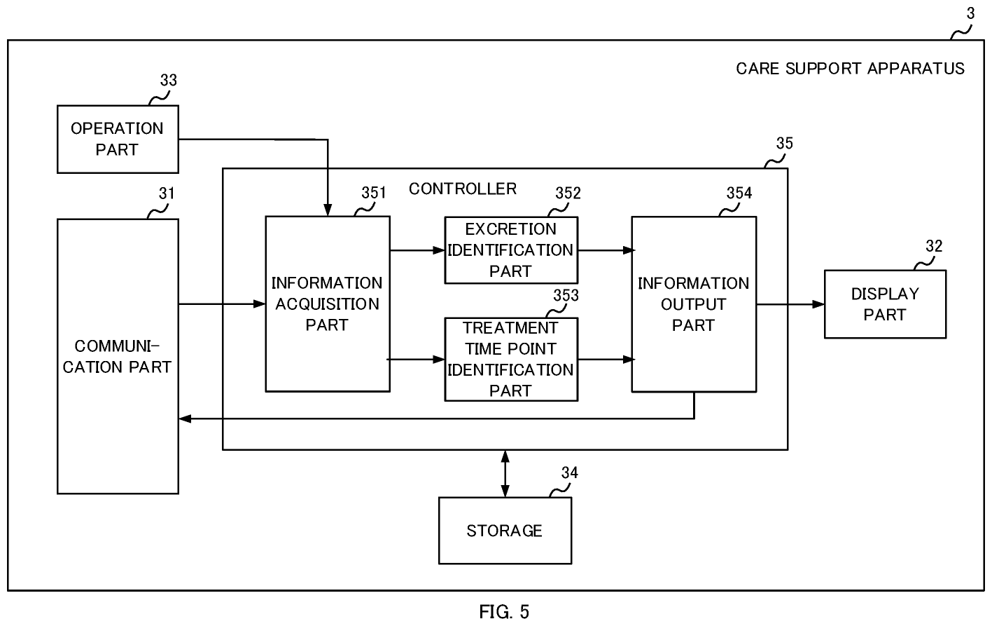

FIG. 5 shows a configuration of the care support apparatus 3. The care support apparatus 3 includes a communication part 31, a display part 32, an operation part 33, a storage 34, and a controller 35. The controller 35 includes an information acquisition part 351, an excretion identification part 352, a treatment time point identification part 353, and an information output part 354.

The communication part 31 is a communication interface for transmitting and receiving information to and from the sensor 1 via the network N. The communication part 31 includes a communication controller for connecting to the network N, for example. The communication part 31 notifies the controller 35 of the odor detection information received from the sensor 1.

The display part 32 is a device for displaying information, and is a display, for example. The display part 32 displays information to be presented to a user of the care support apparatus 3 (for example, an administrator of the care facility or the like).

The operation part 33 is a device for receiving an operation by the user of the care support apparatus 3, and is a mouse, a keyboard, a touch panel, or the like. The operation part 33 inputs a signal indicating the operation contents to the controller 35.

The storage 34 includes a storage medium such as a ROM, a RAM, a hard disk, and the like. The storage 34 stores a program executed by the controller 35, for example. Further, the storage 34 stores a care recipient database including information related to the care recipient and a caregiver database including information related to the caregiver.

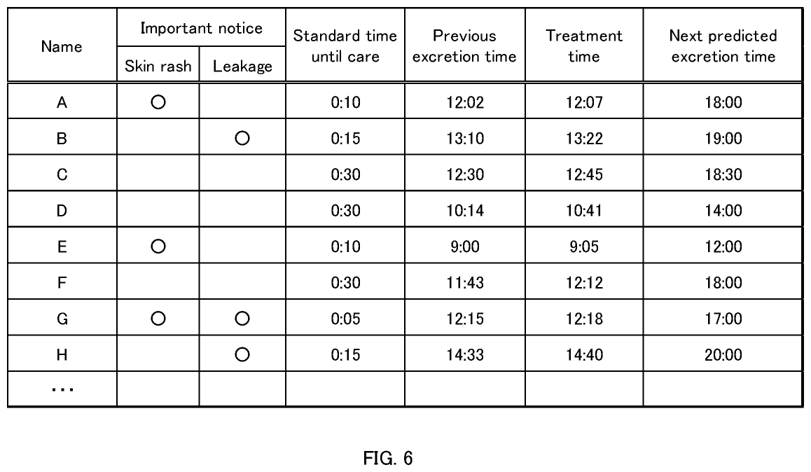

FIG. 6 shows an example of the care recipient database. In the care recipient database shown in FIG. 6, a name of the care recipient, an important notice for each care recipient, a standard time until care, a time of the previous excretion, the last time at which the excretion treatment was performed (treatment time), and a predicted time of next excretion (next predicted excretion time) are associated with each other.

In the important notice column, the care recipient marked with “O” in “Skin rash” indicates that the care recipient is prone to skin rashes caused by excretion, and a care recipient marked with “O” in “Leakage” indicates that the excretion leaks easily from the diaper. Details of the standard time until care will be described later, but it is the time allowed from the excretion time point to the excretion treatment, for example. The standard time until care for the care recipient who falls under the important notice is shorter than the standard time until care for the care recipient who does not fall under the important notice.

Among the data in the care recipient database, a name, an important notice, and a standard time until care are inputted by the administrator of the care facility or the like, for example. Although details will be described later, the previous excretion time, the treatment time, and the next predicted excretion time are registered by the controller 35 on the basis of the data acquired from the sensor 1 and the information terminal 2.

The controller 35 includes a processor such as a CPU. The controller 35 functions as the information acquisition part 351, the excretion identification part 352, the treatment time point identification part 353, and the information output part 354 by executing the program stored in the storage 34.

The information acquisition part 351 acquires various types of information received via the communication part 31. For example, the information acquisition part 351 acquires the odor detection information transmitted by the sensor 1, and notifies the excretion identification part 352 of the acquired odor detection information. Further, the information acquisition part 351 acquires the care treatment performance information transmitted by the information terminal 2, and notifies the treatment time point identification part 353 of the acquired care treatment performance information. The information acquisition part 351 may notify the excretion identification part 352 of the care treatment performance information.

The excretion identification part 352 identifies the excretion time point at which the care recipient excreted, on the basis of the odor detection information notified from the information acquisition part 351. The excretion identification part 352 identifies that the time when the excretion identification part 352 receives the odor detection information indicating that the odor has been detected as the excretion time point, for example. Alternatively, the excretion identification part 352 may identify the excretion time point by analyzing a signal pattern of the odor detection information by using a learned model based on machine learning of the past odor detection information. The excretion identification part 352 notifies the information output part 354 of the identified excretion time point. The excretion identification part 352 may identify a single point (time) on a time axis as the excretion time point, or may identify the excretion time point as a time period with a certain length.

The excretion identification part 352 may further identify the state of excretion at the time of excretion. The excretion identification part 352 identifies whether the type of excretion is feces or urine on the basis of, for example, the information indicating the type of odor component included in the odor detection information. The excretion identification part 352 may further identify the amount of excretion on the basis of the information indicating the intensity of odor indicated by the odor detection information.

When the excretion identification part 352 receives a notification of the care treatment performance information from the information acquisition part 351, the excretion identification part 352 may identify the type of excretion on the basis of the information included in the care treatment performance information. The excretion identification part 352 identifies the type of excretion on the basis of the contents and amount of excretion inputted by the caregiver in the information terminal 2, which is included in the care treatment performance information, for example.

When there are many care recipients in the care facility or the like, the excretion identification part 352 identifies a plurality of excretion times in association with the plurality of care recipients. The excretion identification part 352 stores the excretion time point in the care recipient database in the storage 34, in association with the care recipient.

The treatment time point identification part 353 identifies the treatment time point at which the caregiver performs the treatment of excretion. The treatment time point identification part 353 identifies, for example, that the time at which the treatment time point identification part 353 receives the care treatment performance information indicating that the diaper has been changed is the treatment time point. The treatment time point identification part 353 may identify that the time indicated by the time information included in the care treatment performance information notified from the information acquisition part 351 is the treatment time point. The treatment time point identification part 353 notifies the information output part 354 of the identified treatment time point.

The information output part 354 generates various types of information on the basis of the information notified from the excretion identification part 352 or the treatment time point identification part 353, and outputs the generated information. The information output part 354 generates, on the basis of the past excretion time of the care recipient notified from the information acquisition part 351, the recommendation information indicating when the caregiver of the care recipient is recommended to perform one or more types of care work including the treatment of excretion of the care recipient, and outputs the generated recommendation information. The information output part 354 transmits the recommendation information to the information terminal 2, causes the display part 32 to display the recommendation information, or causes the printer to print the recommendation information.

### First Example of Display Screen of Recommendation Information

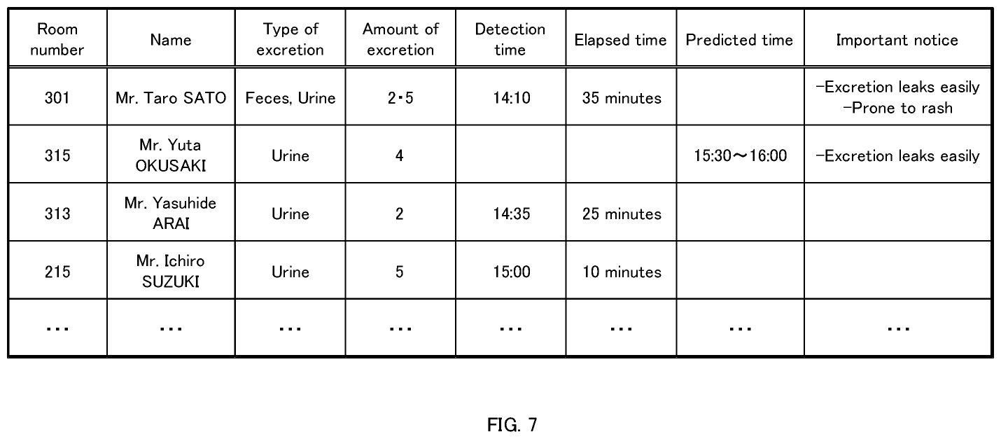

FIG. 7 shows an example of a screen including the recommendation information outputted by the information output part 354. In FIG. 7, a room number, the type and amount of excretion, the time at which the excretion was detected (that is, the excretion time point), a time elapsed from the excretion time point, the time at which the next excretion is predicted, and important notices are displayed in association with each of the plurality of care recipients.

The information output part 354 outputs the time elapsed from the previous excretion time point as the recommendation information in this way, for example. In addition, the information output part 354 identifies the time of day when the care recipient may excrete next on the basis of a plurality of past excretion time points, and outputs the recommendation information indicating the identified time of day. By having the information output part 354 output such recommendation information, the caregiver who sees the recommendation information can know when to change the diaper next, and a manager of the care facility or the like can appropriately assign the caregiver to change the diaper next.

The information output part 354 may generate the recommendation information on the basis of the state of excretion identified by the excretion identification part 352. Specifically, the information output part 354 generates the recommendation information that includes information indicating whether the excretion excreted by the care recipient is feces or urine, and information indicating whether the next predicted excretion is feces or urine. By having the information output part 354 generate such recommendation information, it becomes even easier for the caregiver to plan the care work.

The information output part 354 may output the recommendation information indicating a desirable standard time until care as the time from the point of excretion until performing the treatment of excretion (hereinafter referred to as a “time until care”). The standard time until care is a standard period of time allowed from when the excretion is excreted until completion of changing the diaper, and is stored in the storage 34 in association with the care recipient in advance. The standard time until care for a care recipient who is prone to rashes caused by the excretion and a care recipient whose excretion leaks easily from the diaper is set to be shorter than the standard time until care for other care recipients.

The information output part 354 may output the recommendation information indicating the reason why the excretion should be promptly treated when the standard time until care is equal to or less than the threshold value. The threshold is, for example, an amount of time that the caregiver may need to stop other care work to treat the excretion, and is 15 minutes as an example. If the standard time until care is equal to or less than the threshold value, the information output part 354 outputs the recommendation information that includes, for example, the reason that the care recipient is prone to rashes due to the excretion or that excretion leaks easily from the diaper. By having the information output part 354 output the recommendation information that includes the reason in this way, the caregiver who sees the recommendation information can understand why they should hurry, such that it becomes easier for the caregiver to make appropriate decisions about priorities with work in progress.

The information output part 354 may output the recommendation information indicating trends for excretion of the care recipient on the basis of the plurality of past defecation time points. A tendency of the care recipient's excretion includes, for example, the time of day when the care recipient has a high probability of excretion, and the quality or amount of feces or urine that the care recipient excretes. By having the information output part 354 output such recommendation information, it becomes easier for the caregiver to take an appropriate action after grasping the tendency of the care recipient's excretion.

By referring to the information indicating the past excretion date and time and the past excretion treatment date and time stored in the storage 34, the information output part 354 may determine the standard time until care on the basis of the time elapsed from the past excretion time point to the treatment time point immediately after said past excretion time point. The information output part 354 determines the standard time until care on the basis of, for example, statistical value of the times up to care from the plurality of past excretion time points to the treatment time points. The information output part 354 determines a time that is shorter than the longest time of the plurality of past times until care for a particular care recipient as the standard time until care. For example, the average or median of the plurality of past times until care for the particular care recipient is decided to be the standard time until care. By having the information output part 354 determine the standard time until care, the administrator of the care facility or the like does not have to take time to determine an appropriate standard time until care for each care recipient, such that it is suitable for the care facility or the like with a large number of care recipients.

The information output part 354 may determine the standard time until care on the basis of a treatment time point at which one or more caregivers who satisfy a predetermined condition treated the excretion. The one or more caregivers are experienced caregivers whose experience levels are equal to or greater than the standard level, for example. Experienced caregivers are more likely to understand characteristics of the care recipients, such as whether or not the care recipients are prone to rashes caused by the excretion. Therefore, by having the information output part 354 determine the standard time until care on the basis of the time from the excretion time when the excretion is detected to the treatment time when the experienced caregiver treats the excretion, the standard time until care suitable for the care recipient can be determined.

### Second Example of Recommendation Information Display Screen

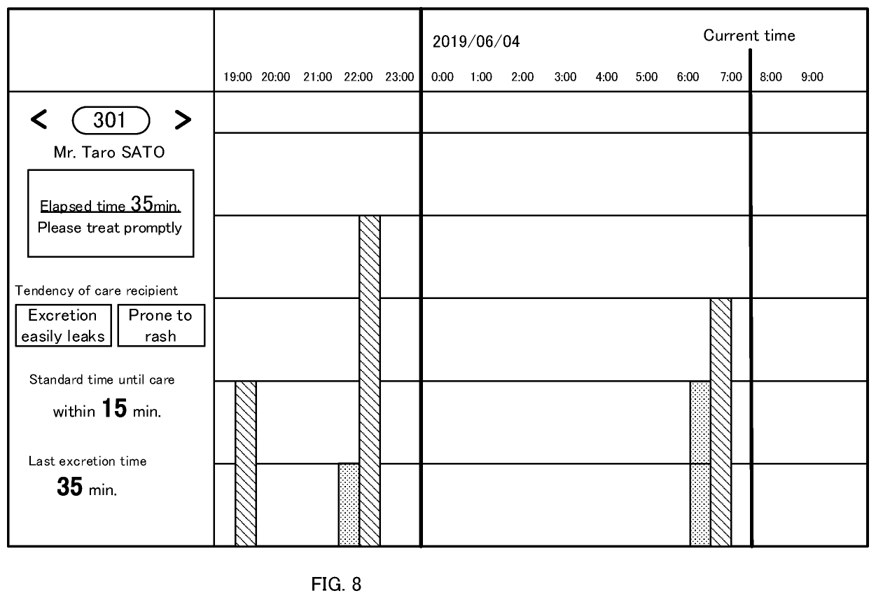

FIG. 8 shows another example of the screen including the recommendation information outputted by the information output part 354. The screen shown in FIG. 8 is displayed on the information terminal 2, for example, when the caregiver selects the care recipient in the screen shown in FIG. 7.

In the screen shown in FIG. 8, the excretion time point, the type of excretion, and the amount of excretion in the last 15 hours for the selected care recipient are shown. The horizontal direction in FIG. 8 indicates time. A rectangle with oblique lines indicates that feces was excreted, and a rectangle with halftone dots indicates that urine was excreted. The length of the rectangle indicates the amount of excretion. The caregiver can easily grasp the time, type, amount, and the like of next excretion by seeing this screen. In addition, this screen also displays the time allowed for treating the excretion and important notices. When the caregivers see these pieces of information, it becomes easier for them to treat the excretion at an appropriate time.

### [Supporting Work for Caring for a Large Number of Care Recipients]

In the care facility or the like, a single caregiver takes care of a large number of care recipients. The plurality of care recipients being cared for by a single caregiver may excrete at different times, or at about the same time. In order for the caregivers to treat the care recipients' excretions in an appropriate order in such an environment, the information output part 354 may generate the recommendation information indicating when the caregiver is recommended to perform the care work for each of the plurality of care recipients on the basis of the plurality of excretion time points of the plurality of care recipients.

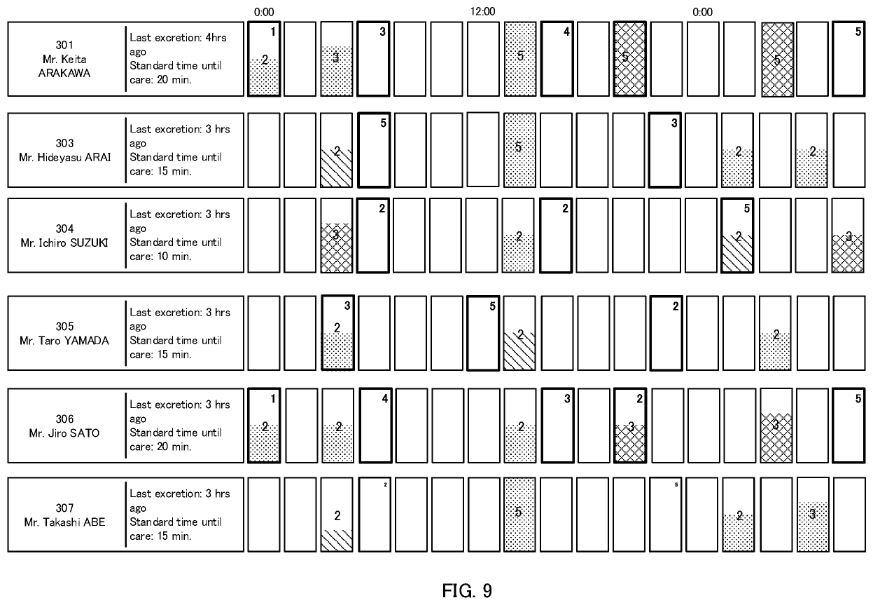

FIG. 9 is an example of a screen that makes it easier for the caregiver to keep track of excretion statuses of the plurality of care recipients. In the screen shown in FIG. 9, the horizontal direction indicates time. In the screen shown in FIG. 9, information indicating the type of excretion excreted by each care recipient is displayed in time series in association with the plurality of care recipients. A thick square indicates that urine is excreted, and an upper right number in the thick square indicates the amount of urine. Squares with halftone dots, oblique lines, halftone lines, and the like indicate that feces are excreted, and the patterns represent color and hardness of the feces. In addition, numbers in the vicinity of the center of the squares indicate the amount of feces. By seeing the screen shown in FIG. 9, the caregiver can easily grasp the excretion status and excretion pattern of the care recipient whom the caregiver takes care of.

The information output part 354 may, for example, generate the recommendation information indicating an order of excretion time points for the plurality of care recipients whom the single caregiver takes care of, and output the generated recommendation information in order to allow the caregiver to grasp information for treating excretion in the order of the excretion time points. The information output part 354, for example, displays a number indicating an order of treatment in association with the care recipient's name on the screen shown in FIG. 9.

Since the care recipients have different standard times until care respectively, the information output part 354 may generate the recommendation information indicating an order of treatment that differs from the order of the excretion time points, on the basis of the excretion time and the standard time until care for each of the plurality of care recipients. Specifically, the information output part 354 subtracts the standard time until care from the time elapsed from the excretion time point to the current time point of each care recipient, and generates the recommendation information indicating the order from the greatest subtraction result. By having the information output part 354 output such recommendation information, the caregiver can prioritize the treatment of excretion of the care recipient who is reaching the deadline of the standard time until care, such that the caregiver is less likely to pass the standard time until care.

### [Supporting Other Care Work]

In the care facility or the like, the caregiver needs to perform various types of care work such as providing a meal, supporting and administering medication, and supporting bathing. Therefore, the caregivers are required to perform these various types of care work for the plurality of care recipients while treating the excretion within the standard time until care. With respect to the provision of the meal, the caregiver needs to provide the meal during the time of day set by the care facility or the like. In such a situation, for example, if the standard time until care is passed while providing the meal, the care recipient's skin may become irritated or the excretion may leak.

In order to support such caregivers, the information output part 354 may output the recommendation information indicating the time when it is recommended to treat the excretion and provide the meal to the care recipients whom the caregiver takes care of, as the care work. For example, the information output part 354 outputs the recommendation information indicating, as the time when the meal should be provided, the time of day when there is low possibility of exceeding the standard time until care even when the time required for providing the meal elapses (for example, 20 minutes).

Specifically, the information output part 354 outputs information indicating, as the time period during which the meal can be provided, a time period in which a time obtained by adding the time required to provide the meal to the time elapsed from the excretion time point of each care recipient to the current time point does not exceed the standard time until care, in the time of day when the meal can be provided. By having the information output part 354 operate in this manner, the caregiver can treat excretion within the standard time until care and provide the meal at an appropriate time of day.

It should be noted that, if there is a care recipient for whom the time obtained by adding the time required to provide the meal to the elapsed time exceeds the standard time until care, the information output part 354 outputs the recommendation information indicating that the treatment of excretion of the care recipient is to be performed first. If the information output part 354 determines that the treatment of the plurality of care recipients' excretion by a single caregiver will not allow the care recipients to be fed in the time of day during which the meal can be provided, the information output part 354 may transmit information requesting support to the information terminal used by the administrator of the care facility or the like. By having the information output part 354 operate in this manner, even if a large number of care recipients excrete immediately before the meal, the treatment of excretion is performed within the standard time until care, and the caregiver can provide the meal at the appropriate time of day.

With regard to medication, the contents of medication (that is, the type of medication and method of administration) and the time of administration vary depending on the care recipient. Therefore, the information acquisition part 351 may further acquire medication information including the contents and time of medication for the care recipient, and the information output part 354 may generate the recommendation information on the basis of the medication information. Since adequate hand washing is required when the caregiver supports the medication after the treatment of excretion, it is desirable that the caregiver continuously support the medication to the plurality of care recipients as much as possible, as with the provision of the meal.

In order for the caregiver to efficiently treat the excretion and support the medication, the information output part 354 first extracts the care recipients whose medication times overlap among the plurality of care recipients whom the caregiver takes care of, and then calculates the times required for administering the medication to the extracted care recipients. The information output part 354 outputs the recommendation information indicating, as a time period in which the medication is to be administered, a time period in which the time obtained by adding the calculated time required for medication to the time elapsed from the excretion time point of each care recipient to the current time point does not exceed the standard time until care. By having the information output part 354 operate in this manner, the caregiver can treat the excretion within the standard time until care, and administer the medicine to the care recipient in an appropriate time of day.

If there is the care recipient for whom the time obtained by adding the time required for administering the medication to the elapsed time exceeds the standard time until care, the information output part 354 outputs the recommendation information indicating that the treatment of the care recipient's excretion is to be performed first. If the treatment of the plurality of care recipients' excretions by the single caregiver will not allow the care recipients to administer the medication in the time of day when the medication should be administered, the information output part 354 may transmit the information requesting support to the information terminal used by the administrator of the care facility or the like. By having the information output part 354 operate in this manner, even if a large number of care recipients excrete immediately before the medication, the excretion is treated within the standard time until care, and the caregiver can administer the medication in an appropriate time of day.

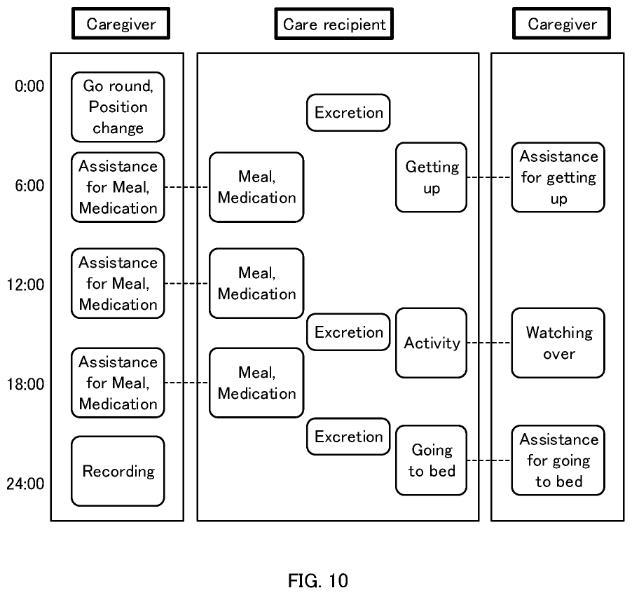

FIG. 10 shows an example of a daily activity plan for the care recipient and caregiver outputted by the information output part 354. The items listed in the column of “caregiver” in FIG. 10 indicate the work to be performed by the caregiver during each hour of the day, and the items listed in the column of “care recipient” indicate the activities to be performed by the care recipient. The “excretion” is shown in the time of day when the care recipient is most likely to excrete. The “meal, medication,” “getting up,” “activity,” and “going to bed” are shown in the recommended times of day for the care recipient to eat, take medication, wake up, participate in an activity, and sleep. By having the information output part 354 output the caregiver's work schedule optimized to match the care recipient's daily rhythm in this way, the caregiver's work can be improved efficiency and the care recipient's satisfaction can be enhanced.

In the above embodiments, an example of optimizing the timings of medication and meals mainly according to the excretion time point has been described, but the contents of the (care) work may be changed on the basis of the excretion time point or treatment time point of each care recipient. For example, the information output part 354 outputs the recommendation information indicating “administering a laxative” to the care recipient who is understood to have “a tendency to be constipated” from the data of the past excretion time point and the like. In addition to this, the information output part 354 may also output the recommendation information indicating a change in the contents of the work performed by the caregiver for the care recipient, such as “increasing the amount of fluid intake,” “revising the contents of the meal such as increasing the amount of dairy products,” and “revising the type or amount of laxative regularly used before administering a laxative.”

Further, the information output part 354 may take into account constraints of facility management (costs, operating rules, and the like) in changing the timing or contents of work. For example, if the information output part 354 obtains the recommendation information indicating that the meal contents should be revised, but the above constrains (for example, the budget limit of the facility) cannot be met considering the cooking cost (cost of materials, labor and time involved in making the meal with a different menu) associated with changing the meal contents, the information output part 354 may output a warning message indicating this fact together with the recommendation information, or output an alternative (changing the meal to a lower cost menu) as the recommendation information.

### [Optimization of Work Sharing Among a Plurality of Caregivers]

There are the plurality of caregivers in the care facility or the like, and the plurality of caregivers share the care work of caring for the plurality of care recipients. Traditionally, the manager of the care facility or the like has decided which of several caregivers takes care of the care recipient, but if several care recipients whom a single caregiver takes care of often excrete at the same time, it may become difficult for the caregiver to treat the excretion within the standard time until care, or there may be times when the caregiver is busy and times when the caregiver is not.

Therefore, the information output part 354 may output the recommendation information that is associated with the plurality of caregivers and indicates when it is recommended to perform the care work for each of the plurality of care recipients. For example, the information output part 354 refers to the history of past excretion time points of all care recipients receiving care at the care facility or the like to predict the time of day when the care work, which is treating the excretion, of each care recipient will occur. The information output part 354 then adds the standard time until care of each care recipient to the predicted time of day to predict an available treatment time period during which the excretion may be treated.

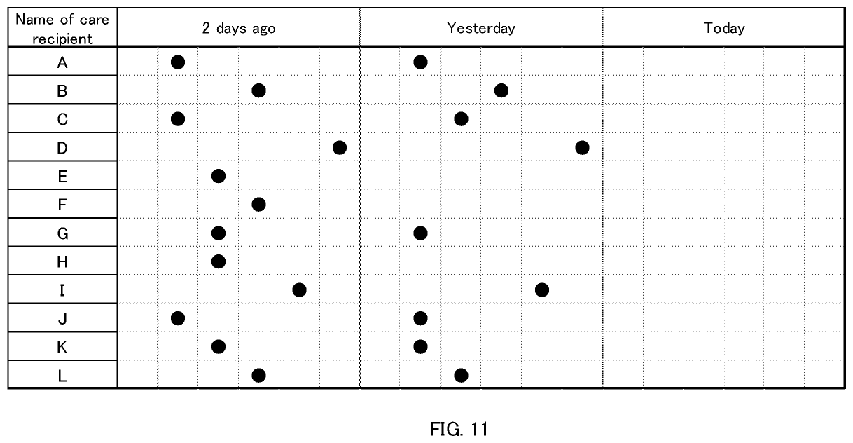

FIG. 11 shows a history of the excretion times of the plurality of care recipients. A black dot in FIG. 11 indicates a time point when a care recipient excreted during the past two days.

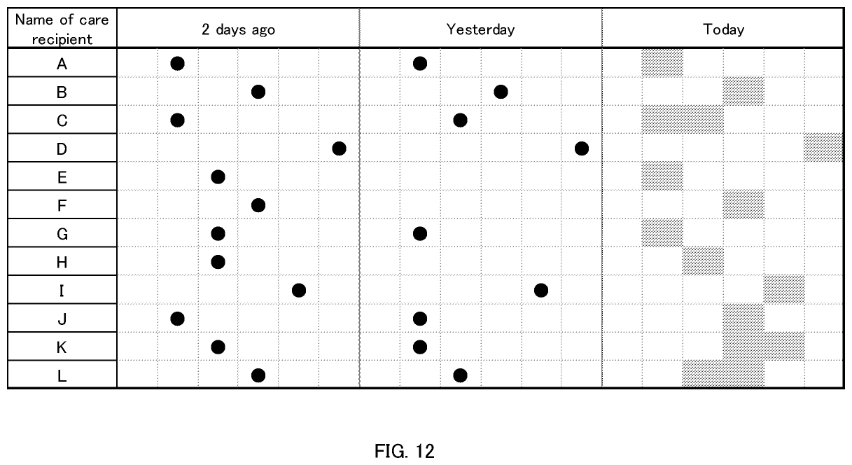

FIG. 12 shows the time of day when each care recipient is likely to excrete. A gray time period in FIG. 12 indicates the time period when each care recipient has a high possibility of excretion, which is identified on the basis of the pattern of past excretion time points.

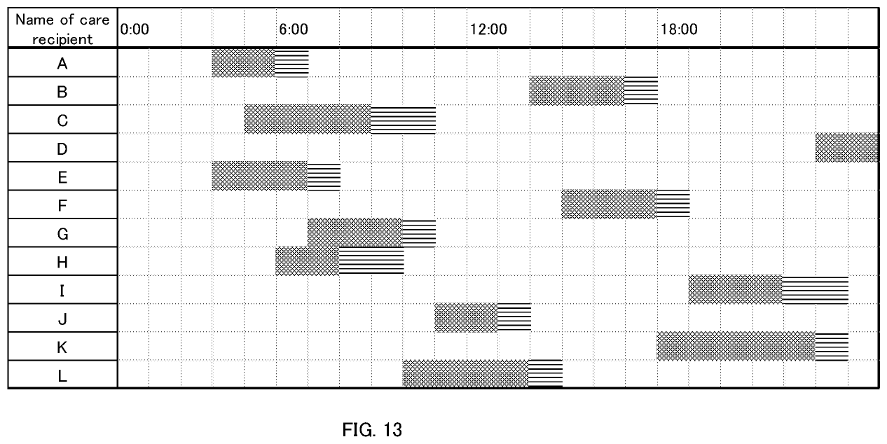

FIG. 13 shows the available treatment time for each care recipient. The time periods with halftone dots in FIG. 13 correspond to the gray time periods in FIG. 12. The time periods with vertical lines in FIG. 13 indicate the standard time until care. The time periods obtained by combining the time periods with halftone dots and the time periods with vertical lines represent the time periods when there is a high possibility that excretion treatment will be required for each care recipient. As described above, the information output part 354 determines the available treatment time shown in FIG. 13 by adding the standard time until care of each care recipient to the gray time period shown in FIG. 12.

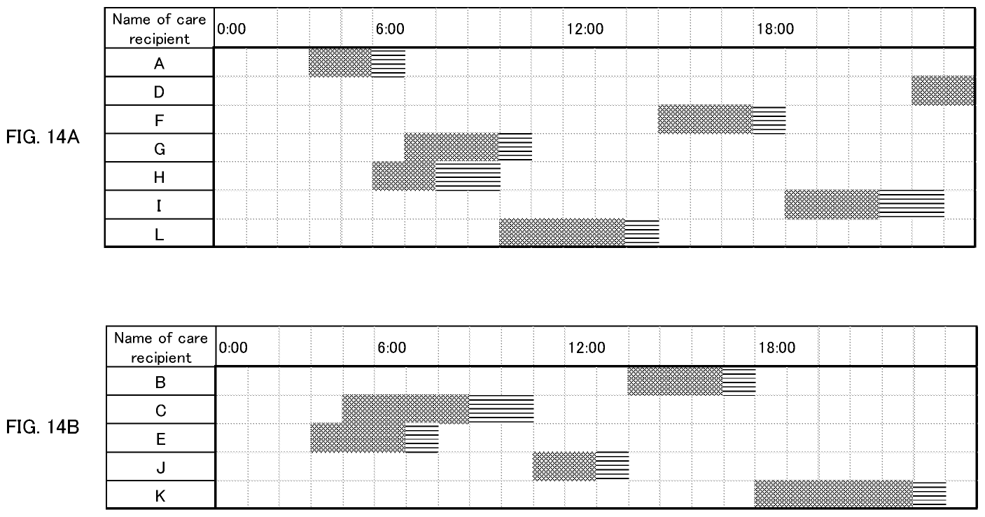

FIGS. 14A and 14B show the available treatment time for each care recipient assigned to the plurality of caregivers. FIG. 14A shows the available treatment times of the plurality of care recipients assigned to a first caregiver, and FIG. 14B shows the available treatment times of the plurality of care recipients assigned to a second caregiver.

The information output part 354 assigns the plurality of care recipients to the plurality of caregivers such that (a) the plurality of available treatment times corresponding to the plurality of care recipients assigned to a single caregiver are distributed as much as possible and (b) a single caregiver can treat a plurality of care recipients' excretion within the standard time until care. Specifically, the information output part 354 assigns the plurality of care recipients to the plurality of caregivers such that the time when the available treatment times of the plurality of care recipients assigned to the each of the caregivers overlap is as short as possible.

Further, the information output part 354 may display, on the information terminal 2 of each caregiver, the order of treating the excretion of the care recipients assigned to each of the plurality of caregivers. By having the information output part 354 output the recommendation information including such contents to the information terminal 2, the possibility that the plurality of caregivers can treat the excretions within the standard time until care of the plurality of care recipients is increased.

The information output part 354 may determine the plurality of care recipients to be assigned to the plurality of caregivers in consideration of the time of day when care work other than the treatment of the excretion is to be performed. In this case, the information output part 354 selects the plurality of care recipients to be assigned to each of the caregivers such that it is not necessary to treat the excretion in a time of day for care work such as providing a meal or supporting medication.

By having the information output part 354 (a) generate the work plan, by selecting the care recipient, such that the times during which the plurality of types of care work to be performed do not overlap as much as possible, and (b) output the generated work plan, it is possible for the plurality of caregivers to perform the various types of care work at appropriate times.

In this case, the information output part 354 may assign the time when the caregiver is recommended to perform the care work within a time period in which the caregiver is allowed to perform the care work in the facility to which the caregiver belongs (for example, a time period other than a break time). By having the information output unit 354 operate in this way, the caregiver can treat the excretion within the standard time until care of the care recipient while taking an appropriate break, since the caregiver's care work is not assigned during the caregiver's break time.

Further, the information output part 354 may generate the recommendation information so as to satisfy a staffing standard set for the care facility to which the caregiver belongs. The staffing standard is defined by the Ministry of Health, Labor and Welfare as the minimum number of nursing staff, care workers, life consultants, full-time administrators, and the like that should be assigned to a care facility or the like. Alternatively, the information output part 354 may determine the plurality of care recipients to be assigned to the plurality of caregivers without considering the staffing standard at the beginning, and then determine whether or not said assignment meets the staffing standard, and output a warning (alert) if the assignment result does not meet the staffing standard. At this time, the information output part 354 may output the warning (alert) if the above assignment results in too many staff members compared to the staffing standard.

### [First Variation]

In the above description, the case where the care support apparatus 3 is installed in the care facility or the like is exemplified, but the configuration and the installation location of the care support apparatus 3 are not limited to the above example.

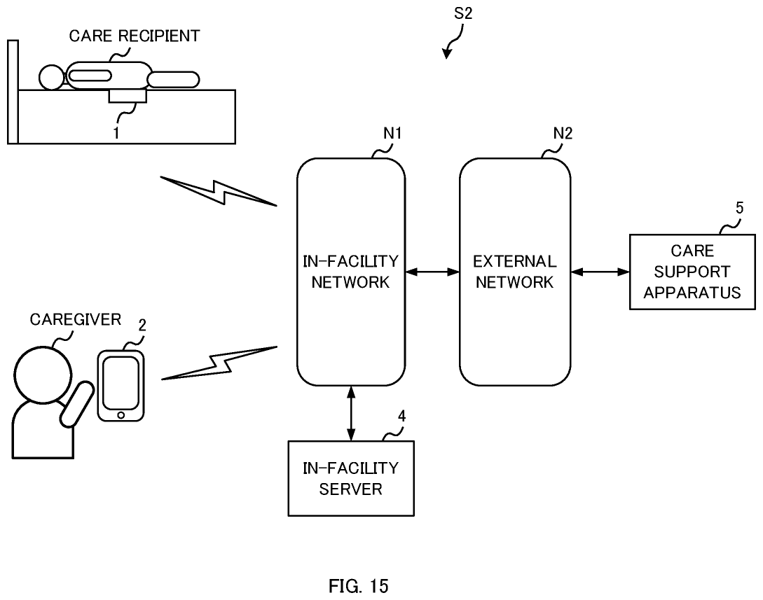

FIG. 15 shows a configuration of a care support system S2 according to a first variation. In the care support system S2, the sensor 1, the information terminal 2, and an in-facility server 4 are connected to an in-facility network N1. The in-facility server 4 is a server that manages personal information of the caregivers, for example. The information terminal 2 and the in-facility server 4 can transmit and receive various types of data to and from a care support apparatus 5 via an external network N2 (for example, the Internet). The care support apparatus 5 is a cloud server having a function equivalent to that of the care support apparatus 3 shown in FIG. 1.

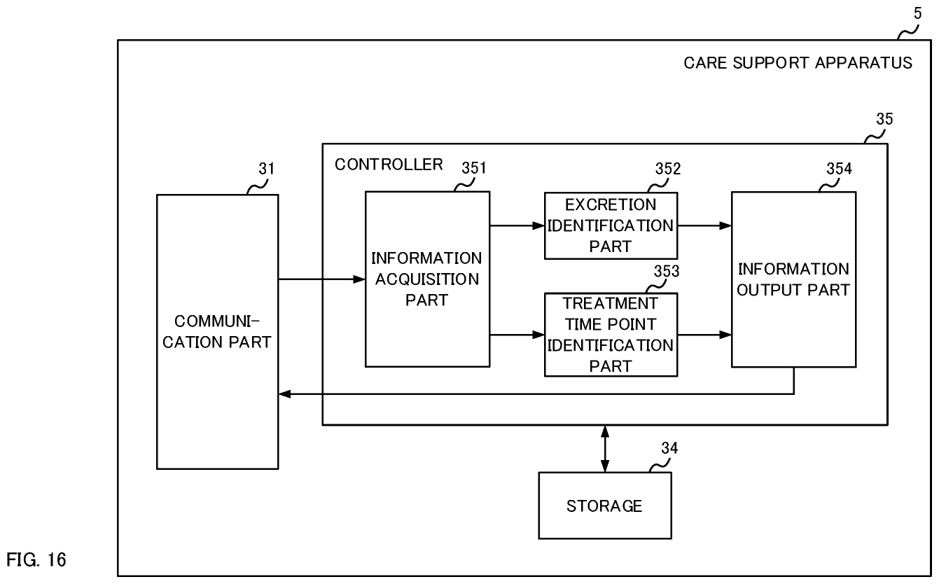

FIG. 16 shows a configuration of the care support apparatus 5. In FIG. 16, components having the same functions as those of the care support apparatus 3 shown in FIG. 5 are denoted by the same reference numerals as those in FIG. 5. The care support apparatus 5 includes the communication part 31, the storage 34, and the controller 35. The controller 35 functions as the information acquisition part 351, the excretion identification part 352, the treatment time point identification part 353, and the information output part 354 by executing the program stored in the storage 34.

When the care support apparatus 5 is realized by a cloud server accessible by the information terminal 2 via the external network N2 in this way, for example, the caregiver can check the recommendation information even while the caregiver is taking the care recipient outside the care facility or the like, and therefore, it is suitable for the case where the caregiver may work outside of the care facility or the like.

### [Second Variation]

In the above description, the treatment time point identification part 353 identifies the treatment time point on the basis of the care treatment performance information indicating that the treatment of the excretion has been performed, but the treatment time point identification part 353 may identify the treatment time point using other means. For example, the treatment time point identification part 353 may determine the time point at which the intensity of the odor detected by the sensor 1 is reduced, as the treatment time point at which the excretion is treated.

However, when the care recipient leaves the bed, the sensor 1 may detect that the intensity of the odor is reduced even though the treatment of the excretion of the care recipient is not performed. Therefore, the information acquisition part 351 may acquire the odor change information indicating the change in odor detected by the care support apparatus 3 installed in the bed of the care recipient or in the vicinity of said bed, and the position information indicating the position of the care recipient detected by a position sensor installed in the bed or in the vicinity of said bed. The odor change information is information that includes text data indicating either that there has been a change in odor or at least the amount of change in odor, for example. In this case, the treatment time point identification part 353 identifies, as the treatment point, a time point at which the odor is determined to have weakened on the basis of the odor change information while the position information indicates that the care recipient is on the bed.

By having the information acquisition part 351 and the treatment time point identification part 353 operate in this manner, the treatment time point identification part 353 can identify the treatment time point without the caregiver having to input that the excretion has been treated into the information terminal 2, thus reducing the load on the caregiver. Further, even if the caregiver forgets to input to the information terminal 2 that the excretion has been treated, the treatment time point identification part 353 can identify the treatment time point.

### [Third Variation]

In the above description, the sensor 1 is an odor sensor for detecting the odor, but the sensor 1 may be a sensor other than the odor sensor. For example, the sensor 1 may be a temperature sensor for detecting the temperature of the diaper or a humidity sensor for detecting the humidity, and may detect the excretion on the basis of changes in temperature or humidity.

### [Fourth Variation]

In the above description, a case where the care support apparatus 3 can be realized by a computer installed in the care facility or the like has been exemplified, but the functions of the care support apparatus 3 may be realized by a processor installed in the sensor 1 and the information terminal 2 by executing application software.

The present invention is explained on the basis of the exemplary embodiments. The technical scope of the present invention is not limited to the scope explained in the above embodiments and it is possible to make various changes and modifications within the scope of the invention. For example, all or part of the apparatus can be configured with any unit which is functionally or physically dispersed or integrated. Further, new exemplary embodiments generated by arbitrary combinations of them are included in the exemplary embodiments of the present invention. Further, effects of the new exemplary embodiments brought by the combinations also have the effects of the original exemplary embodiments.

What is claimed is:

1. A care support apparatus comprising:
an excretion identification part that identifies an excretion time point at which a care recipient excretes on the basis of information from a sensor provided in the vicinity of the care recipient; and
an information output part that generates recommendation information indicating when a caregiver of the care recipient is recommended to perform one or more types of care work including treatment of the excretion of the care recipient on the basis of past excretion time points of the care recipient, and outputs the generated recommendation information.

2. The care support apparatus according to claim 1, wherein
the information output part outputs a time elapsed from a previous excretion time point as the recommendation information.

3. The care support apparatus according to claim 1, wherein
the information output part identifies a time period in which the care recipient may excrete next on the basis of a plurality of the past excretion time points, and outputs the recommendation information indicating the identified time period.

4. The care support apparatus according to claim 1, wherein
the excretion identification part further identifies a state of the excretion at the excretion time point, and
the information output part generates the recommendation information on the basis of the state identified by the excretion identification part.

5. The care support apparatus according to claim 1, wherein
the information output part outputs the recommendation information indicating a standard time until care that is desirable as a time from the excretion time point until the excretion is treated.

6. The care support apparatus according to claim 5, wherein
the information output part outputs the recommendation information indicating a reason why the excretion should be treated promptly if the standard time until care is equal to or less than a threshold value.

7. The care support apparatus according to claim 5, further comprising:
a treatment time point identification part that identifies a treatment time point at which the caregiver performs the treatment of the excretion, wherein
the information output part determines the standard time until care on the basis of a time elapsed from the past excretion time point to the treatment time point immediately after the excretion time point.

8. The care support apparatus according to claim 7, wherein
the information output part determines the standard time until care on the basis of the treatment time point at which one or more of a plurality of the caregivers who satisfy a predetermined condition performed the treatment of the excretion.

9. The care support apparatus according to claim 7, further comprising:
an information acquisition part that acquires odor change information indicating a change in odor detected by an odor sensor installed in the bed of the care recipient or in the vicinity of the bed, and position information indicating the position of the care recipient detected by a position sensor installed in the bed or in the vicinity of the bed, wherein
the treatment time point identification part identifies, as the treatment time point, a time point at which it is determined that the odor is weakened on the basis of the odor change information while the position information indicates that the care recipient is on the bed.

10. The care support apparatus according to claim 1, wherein
the information output part outputs the recommendation information indicating a tendency related to the excretion of the care recipient on the basis of the plurality of the past excretion time points.

11. The care support apparatus according to claim 1, further comprising:
an information acquisition part that acquires medication information indicating contents and time of medication for the care recipient, wherein
the information output part generates the recommendation information further on the basis of the medication information.

12. The care support apparatus according to claim 1, wherein
the excretion identification part identifies a plurality of the excretion time points in association with a plurality of the care recipients, and
the information output part generates the recommendation information indicating when the caregiver is recommended to perform the care work for each of the plurality of care recipients on the basis of the plurality of excretion time points of the plurality of care recipients.

13. The care support apparatus according to claim 12, wherein
the information output part outputs, in association with the plurality of care recipients, the recommendation information indicating when the caregiver is recommended to perform the care work for each of the plurality of care recipients.

14. The care support apparatus according to claim 1, wherein
the information output part outputs, as the care work, the recommendation information indicating times at which it is recommend to treat the excretion and provide a meal to the care recipient.

15. The care support apparatus according to claim 1, wherein
the information output part assigns a time at which the caregiver is recommended to perform the care work within a time period during which the caregiver is allowed to perform the care work in a facility to which the caregiver belongs.

16. The care support apparatus according to claim 1, wherein
the information output part generates the recommendation information so as to satisfy a staffing standard set for a care facility to which the caregiver belongs.

17. A care support method comprising the steps, executed by a computer, of:
identifying an excretion time point at which a care recipient excretes on the basis of information from a sensor provided in the vicinity of the care recipient;
generating recommendation information indicating a time at which a caregiver of the care recipient is recommended to perform one or more types of care work including treatment of excretion of the care recipient on the basis of the past excretion time point of the care recipient; and
outputting the generated recommendation information.

18. A care support system comprising:
an information terminal that is capable of displaying information; and
a care support apparatus that outputs recommendation information to the information terminal, wherein
the care support apparatus includes:
an excretion identification part that identifies an excretion time point at which a care recipient excretes on the basis of information from a sensor provided in the vicinity of the care recipient, and
an information output part that generates recommendation information indicating a time at which a caregiver of the care recipient is recommended to perform one or more types of care work including treatment of excretion of the care recipient on the basis of past excretion time point of the care recipient, and outputs the generated recommendation information, and
the information terminal includes:
a display part that displays the recommendation information.
